(19) United States
(12) Patent Application Publication
(10) Pub. No.: US 2021/0196661 A1
(43) Pub. Date: Jul. 1, 2021

(54) METHOD FOR AMELIORATION OF INSULIN RESISTANCE

(71) Applicant: JOSEPH MICHAEL ABRAMOWITZ, SAN DIEGO, CA (US)

(72) Inventor: JOSEPH MICHAEL ABRAMOWITZ, SAN DIEGO, CA (US)

(21) Appl. No.: 17/137,216

(22) Filed: Dec. 29, 2020

Related U.S. Application Data
(60) Provisional application No. 62/954,821, filed on Dec. 30, 2019.

(51) Int. Cl. A61K 31/198; A61K 31/155; A61K 31/4439; A61K 45/06; A61P 3/10

(52) U.S. Cl. CPC A61K 31/198; A61K 31/155; A61P 3/10; A61K 45/06; A61K 31/4439

(57) ABSTRACT

A method is disclosed utilizing the administration of extended release L-tri-iodothyronine in order to ameliorate insulin resistance. In other embodiments extended release L-tri-iodothyronine is combined with a biguanide and/or a thiazolidinedione and/or an inhibitor of the endoplasmic reticulum integrated stress response to provide a multi-targeted and synergistic approach to ameliorate insulin resistance.

### RELATED APPLICATIONS

This application claims the benefit of priority of U.S. Provisional Application Ser. No. 62/954,821 filed Dec. 30, 2019 of common inventorship and which is hereby incorporated by reference in its' entirety.

### FIELD OF THE INVENTION

The present invention relates to insulin resistance and, more particularly, to methods for preventing or reversing the condition.

### BACKGROUND

Insulin resistance (IR) is a resistance manifested by cells to the effects of the hormone insulin. The cells behave in a manner suggesting that they are experiencing an insulin deficit when in fact the extracellular levels of insulin are elevated, often markedly so. The mechanisms underlying IR are not well understood and are the subject of debate. IR is found in numerous and diverse pathologic conditions including, most notably, the metabolic syndrome, type 2 diabetes mellitus (T2DM), Alzheimers disease, atherosclerotic vascular disease and the medical complication of pregnancy known as pre-eclampsia. IR constitutes an escalating global epidemic, particularly in first world countries where humans are exposed to excess caloric intake and other adverse lifestyle factors.

In 1921 Banting, Best and colleagues discovered insulin. The first clinical use of insulin took place in 1922. The concept of insulin resistance was first proposed by Falta in 1931. Himsworth confirmed this hypothesis in a landmark study published in 1936. Himsworths' conclusion was that there were two types of diabetes mellitus. Diabetes mellitus caused by pancreatic failure resulting in insulin deficiency, now known as type 1 diabetes mellitus (T1DM), occurred in younger patients while diabetes mellitus caused by insulin resistance, now known as type 2 diabetes mellitus (T2DM) occurred in older patients who were generally obese.

Insulin resistance is usually measured in the following ways: the glucose clamp method is the reference standard for IR measurement but is laborious. Simpler alternatives are the HOMA test and the QUICKI test. The HOMA (Homeostasis Model Assessment) test is based on a model of interactions between glucose and insulin dynamics that is then used to predict fasting steady-state glucose and insulin concentrations for a wide range of possible combinations of insulin resistance and beta cell function. The HOMA index is derived by an equation directed to determining a surrogate index of insulin resistance. This equation is described by the product of the fasting glucose and the fasting insulin, divided by a constant of 22.5. This constant represents an idealized concept of normal insulin sensitivity. The QUICKI (Quantitative Insulin Sensitivity Check Index) index is an empirically derived mathematical transformation of fasting blood glucose and plasma insulin concentrations that provides an accurate index of insulin sensitivity. QUICKI is calculated by the equation: 1/Log (Fasting Insulin in uU/ml)+Log (Fasting Glucose in mg/dl).

Environmental risk factors for T2DM are well understood. It is generally accepted that excessive caloric intake is a prerequisite for T2DM. The general lack of regular rigorous exercise in these patients is also significant. Genetic risk factors, which play a very significant role, are less well understood. Many gene polymorphisms have been identified as being associated with T2DM.

Prediabetes (PreDM) is a term coined in the 20^{th} century in an attempt to predict at risk patients who were expected to progress to overt T2DM. More recently there has been a strategy involving placing some of these patients on anti-diabetic medications in an attempt to prevent the development of overt T2DM. From the inception of the concept of PreDM, up to the present time, the yardsticks used to diagnose PreDM have had poor predictive value. Currently in the United States patients who are found to have a hemoglobin A1C (HbA1C) level greater than 5.6%, but not high enough to be considered overtly diabetic, are labeled by the testing laboratories as having PreDM. A significant percentage of these patients will never go on to develop overt T2DM. From the time of diagnosis of PreDM, approximately 25% of patients will progress to overt T2DM over the next 5 years. Over the ten years following diagnosis, this number increases to around 50%. In those prediabetic patients not destined to progress to overt T2DM, aside from sensible lifestyle modification, it should be agreed that no antidiabetic medication is necessary.

In regard to thyroid hormone (TH) metabolism, the past few decades have heralded much research into and an understanding of the iodothyronine deiodinase (DI) enzymes whose job it is, in space and time, to defend the optimum balance of TH. The balance of thyroid hormone (BoTH) is defined as a space and time dependent phenomenon whereby a precise degree of TH activation or inactivation is called for and achieved. Peripheral blood measurement of thyroid stimulating hormone (TSH), L-tri-iodothyronine (T3) and L-thyroxine (T4) are examples of the so-called thyroid function tests and they are tests reflecting thyroid hormone kinetics. The term kinetics refers to what the body does to thyroid hormone, regulating its' manufacture and transport. These tests tell little about the adequacy of the executive function of TH in the target tissues, which lies in the domain of TH dynamics. The term dynamics refers to what TH does to the body. There are no blood tests in current clinical usage which can confirm adequacy of these executive functions. Anomalies in TH dynamics may exist in the face of normal TSH and free T4 levels. Thus anomalies may exist which impact what TH does to the body without being reflected in routinely ordered thyroid blood tests.

Controversy exists over which TH replacement therapy is best, whether it is T4 monotherapy or the T3/T4 combination exemplified by desiccated thyroid (DT). The current generation of thyroidologists and their expert committees, both in the United States and in Europe, have voiced concerns regarding the T3/T4 combination therapy as found in DT: (i) They believe correctly that immediate release T3 administration results in absorption spikes in plasma levels of T3 which are supra-physiologic and which put the patient at risk for cardiac arrhythmias; (ii): They point out that batches of animal-sourced DT have inconsistent ratios of T4 to T3; (iii): They allege that the ratio of T4 to T3 found in animal-sourced DT is not the same as the ratio found in humans. Consequently, these expert committees recommend that T4 monotherapy is the standard of care for TH replacement therapy. What is omitted here is that T4 monotherapy results in an increased ratio of T4:T3 in the plasma of the recipient of the pharmaceutical, a phenomenon which is not physiologic and which has been questioned. Elevated T4 blood levels are known to lead to ubiquitination of DI type 2 (D2) {1}. While this ubiquitination reaction is reversible, it inactivates the enzyme, slowing the rate of the activation reaction, which converts T4 to T3.

Further, the efficacy of T4 monotherapy depends upon the capacity of the organism to convert T4 to T3, a phenomenon necessitating normal function of D2. In cases of an inherited polymorphism of D2 and in cases of acquired conditions associated with endoplasmic reticulum (ER) stress (which is associated with reduced D2 activity), such as the metabolic syndrome, type 2 diabetes mellitus, Alzheimers disease, atherosclerotic vascular disease and pre-eclampsia, the activation reaction executed by D2 is substantially reduced.

### SUMMARY

Thyroid hormone is a known insulin sensitizer, although the mechanisms by which TH sensitizes cells to insulin are not clear. The present disclosure claims to understand certain of these mechanisms, as described below. Amelioration of IR by definition results in a reduction of the dose of exogenous insulin required in T2DM. This is axiomatic. A reduction in the exogenous insulin dose required to treat T2DM or the elimination of the requirement for exogenous insulin are goals of the present disclosure. Some patients with T2DM and severe IR are receiving hundreds of units of insulin per day. It has been suggested that these high doses somehow beget further IR and that, beyond a certain number for units per day, higher daily insulin dosages are counterproductive.

It has been suspected since the mid-20^{th} century that endogenous insulin secreted directly into the portal venous system with a ‘first pass’ effect through the liver has subtly differing metabolic effects to those of exogenous insulin absorbed peripherally. Exogenous insulin has also been shown to inhibit pancreatic insulin secretion. This issue remains controversial. However if there is a benefit to pancreatic insulin secretion with its' hepatic first pass effect, this benefit is partially negated by exogenous insulin administration. In the ideal setting it appears optimal for the sole source of insulin in T2DM to be of endogenous pancreatic origin. This too is a goal of the present disclosure.

Historically it has been viewed as illegitimate to use TH to ameliorate IR in diabetic patients considered euthyroid. For reasons relating to two key factors outlined below, this belief is outdated. Not only is the use of ERT3, in judicious doses, as an insulin sensitizer legitimate and justified by the data. It could also be argued that administering ERT3 to patients with the metabolic syndrome and T2DM is a necessity in order to combat one of the most substantial epidemics ever to affect the human race, namely IR. The first of the two factors is: in the outdated paradigm, normal thyroid function tests reflecting normal hypothalamic-pituitary-thyroid-axis (HPTA) function were considered to guarantee normal TH action in peripheral tissues. It is now recognized, with a new paradigm, that under certain circumstances TH actions in the periphery may be uncoupled from the HPTA. This occurs due to local effects of the DI enzymes and other factors affecting TH signaling {1}. The second of the two factors is: ER stress has been found to reduce the activation of TH by D2, resident in the ER {2}. This introduces the significant element of cellular hypothyroidism, only remedied by the administration of T3. These two factors will be expanded upon below. It has been reported that previously undiagnosed hypothyroidism may trigger the clinical onset of T2DM {3}. Hypothyroidism results in ER stress. ER stress is both caused by hypothyroidism and, in cases in which ER stress has a cause other than hypothyroidism, ER stress causes cellular hypothyroidism by the above-referenced mechanism.

Some skilled in the art have a belief, unsupported by data, that administering T3 to euthyroid patients puts them at risk for becoming hyperthyroid. This is incorrect. In a vast majority of euthyroid patients judicious use of ERT3, in doses disclosed here, with clinical and laboratory monitoring is safe. If concerns arise in the occasional patient (as they likely will) the dose of T3 can be lowered and, going forward, a risk/benefit analysis can be conducted. If the T3 therapy fails this test, it is discontinued.

Environmental risk factors for T2DM are well understood. It is accepted that excessive caloric intake is a prerequisite for T2DM. The lack of regular rigorous exercise in these patients is also significant. Genetic risk factors, which play a very significant role, are less well understood. Many gene polymorphisms associated with T2DM have been identified. A given genetic risk factor may not have the power to generate the development of T2DM on its own and the production of the disease may require two or more such genetic risk factors to coexist in a given patient. Many different metabolic aberrations have been associated with T2DM. Whether each is a cause or an effect frequently constitutes uncertainty. The complexity of the metabolic aberrations has led to a reticence to identify a central metabolic villain. This hampers the goal of formulating an effective therapeutic strategy. The exact preclinical pathophysiologic sequence for the progressive development of IR, with reference in the instant case to the metabolic syndrome and T2DM, remains undefined. However the preponderance of the evidence {4} suggests that by the time overt clinical metabolic syndrome and T2DM have been diagnosed, the metabolic disturbance central to IR is oxidative stress (OS) {4}. The cellular organelles most affected by the OS are the ER (ER stress) and the mitochondria (mitochondrial stress).

It is generally accepted in the art that genetic and environmental factors act over time in the development of PreDM. The process is incompletely understood. The art of the present disclosure posits that OS is both the epicenter and the prerequisite for IR in T2DM in both the preclinical and clinical stages and that, with rare exceptions, absent OS there can be no IR or T2DM. Further the present disclosure posits that, while the above-referenced genetic and environmental factors constitute the exclusive etiologies of PreDM, as the ER stress worsens during the pre-clinical phase the impairment in the activity of D2 represents a ‘last straw’ phenomenon and chronicles the entry of the condition onto the clinical horizon. The cellular hypothyroidism resulting from the ER stress is thus the major etiologic factor accounting for the transition from Pre-DM to a clinical diagnosis of overt T2DM. It is believed that this has not been suggested or claimed in publication. This contention, supported by the work by Arroyo e Drigo{2}, that ER stress substantially reduces the activation of TH by D2 in the ER, constitutes the basis for the novelty of the present disclosure and the justification for the administration of T3 to these patients. This is further elaborated upon in FIG. 2.

Arroyo e Drigo {2} found that the above referenced decrease in TH activation was mediated by a eukaryotic initiation factor 2-alpha (eIF2a) mechanism. Under conditions of ER stress one of the mechanisms by which the unfolded protein response (UPR) is initiated is by the triggering of the integrated stress response (ISR) whereby the induction of protein kinase RNA-like endoplasmic reticulum kinase (PERK) as well as other protein kinases occurs. As a result, phosphorylation of eIF2a is increased. This leads to the selective transcription of Activating Transcription Factor 4 (ATF4) and the promotion of a pro-adaptive signaling pathway resulting in the inhibition of global protein synthesis. Krukowski {5} demonstrated that the ISR inhibitor ISM rapidly reverses age-related memory impairment in mice and decreases levels of phosphorylated eIF2a as well as levels of ATF4. The results are extrapolated to the prediction of probable salutary effects with these mechanisms when applied ER stress and ISR inhibition in T2DM.

Most therapeutic approaches to T2DM (biguanides (BG's) and thiazolidinediones (TZD's) excepted) involve attempts to increase the amount of insulin, or insulin effect, to which the cells are exposed. This is achieved by increasing the amount of insulin produced by the pancreatic beta cells, by attempting to enhance insulins' effect or by administering insulin as a drug. No therapeutic intervention in the prior art addresses correction of the metabolic paralysis exemplified by the consequences of OS, as do the methods and formulations of the present disclosure.

The present disclosure involves methods and formulations for novel applications for the administration of ERT3 to human patients for the purpose of ameliorating IR, with particular reference to the metabolic syndrome and T2DM. The method may also ameliorate pathophysiology in other conditions associated with IR, such as Alzheimers disease, atherosclerotic vascular disease and pre-eclampsia. IR is central to the pathophysiology of T2DM, although the role of IR in generating all the features of the metabolic syndrome is less clear. One key effect of ERT3 is to reduce OS in IR and T2DM. OS impairs mitochondrial and ER function in virtually all tissues including the pancreatic beta cells, hepatic cells and peripheral tissues such as adipose tissue, muscle and brain. D2, the chief activator of TH, is resident in the ER. ER stress results in a post-translational downregulation of D2 {2} resulting in suboptimal levels of T3 being produced by the cell. This qualitatively and quantitatively impairs the mitochondria which further increases the OS in what becomes a vicious cycle. This phenomenon represents the impetus for the transition from PreDM to overt T2DM. Administration of ERT3, with or without other drugs targeting IR (in the instant case a BG and/or a TZD and/or an ISR inhibitor) to affected patients may ameliorate IR and may result in a lowered risk of prediabetic patients transitioning to overt diabetes, improvement in the parameters of the metabolic syndrome and improved glycemic control in patients with clinical syndromes of IR. Additional benefits may include reduced dosing requirements for anti-diabetic drugs including insulin and a correction of the cellular hypothyroidism which is implicit to the pathophysiology described above. Further, the art of the present disclosure may reduce the incidence of diabetic complications including, but not limited to, nephropathy, retinopathy, neuropathy and peripheral vascular disease. This is anticipated due to the fact that that poor glycemic control and OS are arguably the two most significant variables leading to these complications. The formulations of the present disclosure may have salutary effects on glycemic control and on oxidative stress. Further, a synergism is anticipated between the elements of the formulation of the instant disclosure in regard to combating IR. This is expanded upon below.

Referenced further below, teleologically the human organism has evolved with a strict mandate to protect itself from unwanted thyroid hormone activation. This is evidenced by the manner in which thyroid hormone is handled in the human embryo as well as in post-natal target tissues {6}. This makes the human organism vulnerable to pathology leading to even small degrees of decreased TH activation.

Thus what is clearly needed is a treatment option comprising methods and therapeutic compositions administered to human patients in order to circumvent the block to the activation of TH and incorporating strategies that use multi-drug and multi-mechanism approaches to the amelioration of the molecular pathology of IR. In so doing the art of the present disclosure may prevent progression of PreDM to overt T2DM, may improve glycemic control in T2DM, may ameliorate elements of the metabolic syndrome, may reduce the incidence of diabetic complications and may reduce the requirement for exogenous insulin and other pharmacologic interventions in T2DM.

### BRIEF DESCRIPTION OF THE DRAWINGS

FIG. 1 is a diagram of type 2 diabetes mellitus presented as the conventional view of diabetes in reverse.

FIG. 2 is a diagram of how cellular hypothyroidism is both a cause and a consequence of oxidative stress.

FIG. 3A is a diagram of endoplasmic reticulum stress without T3 supplementation.

FIG. 3B is a diagram of endoplasmic reticulum stress with T3 supplementation.

FIG. 4A shows the absence of the endoplasmic reticulum integrated stress response under conditions of normal redox balance.

FIG. 4B shows the endoplasmic reticulum integrated stress response triggered by abnormal redox balance.

FIG. 5 shows salutary effects of T3 administration and integrated stress response inhibition on the effects of the integrated stress response.

FIG. 6 is a protocol for transitioning patients with IR to the art of the present disclosure in patients taking exogenous insulin, with TH as the sole therapy.

FIG. 7 is a protocol for transitioning patients with IR to the art of the present disclosure in patients taking exogenous insulin, with TH in combination therapy with a biguanide and/or a thiazolidinedione.

### DETAILED DESCRIPTION

Despite decades of resources invested, IR remains standing in defiance of a comprehensive, clear understanding and in defiance of an effective corrective treatment. Nonetheless, IR found in the metabolic syndrome and T2DM can be summarized as follows: IR develops over time due to chronic caloric excess, the metabolism of which generates OS, overwhelming the redox capacity of the cell, in a patient with a metabolic infrastructure genetically susceptible to the development of the metabolic syndrome or T2DM. The redox imbalance disrupts ER and mitochondrial function. In IR the ER and the mitochondrion are disabled and need to be rehabilitated. It is a goal of the present disclosure to restore ER and mitochondrial function in a manner that no other method, currently accepted and in use for the treatment of IR in the metabolic syndrome or T2DM, addresses.

Throughout the present disclosure there may appear the impression that IR and T2DM are one and the same. They are not. The relationship between the two is as follows: IR is a prerequisite for the development of T2DM. IR precedes T2DM by many years and IR eventually reaches a quantitative severity, after traversing PreDM, whereby overt clinical, diagnosable T2DM is present. In patients who have progressive IR but who do not develop overt clinical, diagnosable T2DM, the presumption is made that these patients have been blessed with genetic factors which protect them from overt clinical, diagnosable T2DM. IR should be viewed as a single iceberg with multiple zones protruding above the water. In the universe of human disease, the most significant of these unsubmerged pieces are the metabolic syndrome, T2DM, Alzheimers disease, atherosclerotic vascular disease and pre-eclampsia.

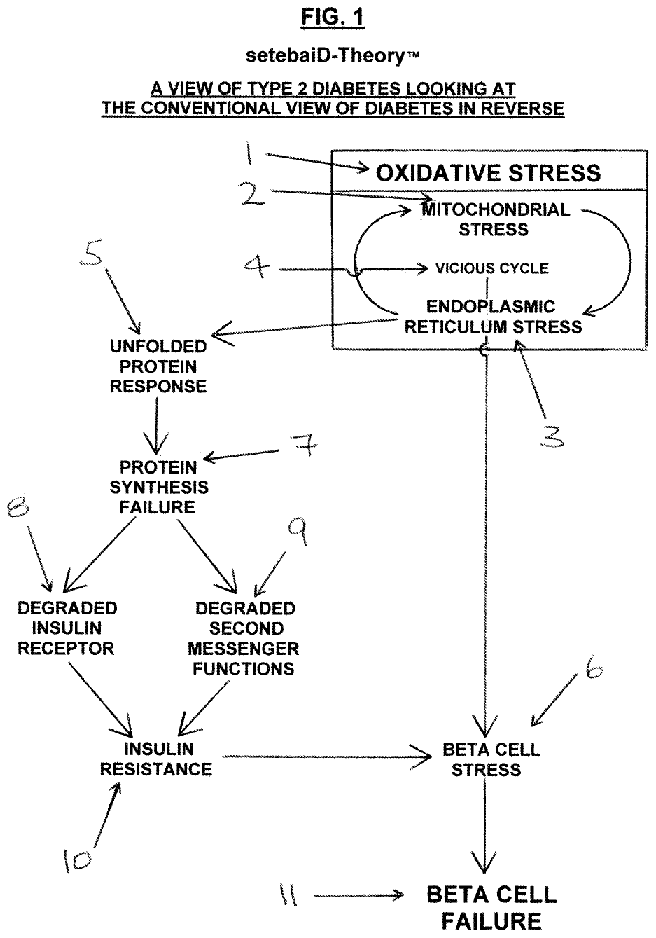

A reticence to define the epicenter of IR compromises the ability to find solutions and therapeutic approaches. Taking the step of defining the epicenter of IR thus serves a constructive purpose. With T1DM this is an easy task as it is well accepted that the origin of T1DM is beta cell failure. While T2DM gives the appearance of being T1DM in reverse, this has not been formally codified. The ‘setebaiD-Theory’, shown below in FIG. 1, postulates that in regard to the path to the diabetic state T2DM is T1DM in reverse and it defines the epicenter of IR in the metabolic syndrome and T2DM as being OS {4}. OS in the ER leads to ER stress which triggers the UPR. The UPR is a natural cellular stress response related to and triggered by ER stress. It is believed conserved in all mammalian species. The UPR aims at restoring normal function to the cell by halting protein translation, degrading misfolded proteins and activating signaling pathways involved in normal protein folding. In the event that these objectives are not achieved within a certain time frame, the UPR shifts its goal to apoptosis by promoting cell death. A component of the UPR is the ISR. This involves activation PERK and other protein kinases. PERK hyper-phosphorylates eIF2a which then switches on the ATF4 gene, activating ATF4 and halting protein synthesis. Further, based on current evidence, PERK pathway activation by the UPR, with its' attendant phosphorylation of eIF2a and activation of the ATP4 pathway, constitutes the epicenter of the epicenter of IR.

T1DM and T2DM have long been known to be two distinct and separate diseases. However, their similarities have been underappreciated until recently. While beta cell failure occurs early in T1DM (as opposed to occurring later in T2DM), OS may be present throughout the natural history of T1DM {7}. It occurs early in the beta cells as they struggle to survive the autoimmune insult that initiates the disease. As T1DM progresses OS also occurs in the cells of tissues which are destined to suffer diabetic complications, such as nerve, retina, kidney and vascular tissue. Further, some patients with T1DM have a tendency to develop features of obesity which include the macro (fat distribution) and the micro (the biochemistry of obesity). Thus patients with T1DM may develop OS, a key feature of IR and T2DM.

Patients with T2DM generally do not develop T1DM. There is one rare exception to this, namely the entity known as latent autoimmune diabetes of adulthood (LADA). When there is a co-occurrence of T2DM and LADA in the same patient, it is probable that these are truly two separate diseases. It is important to note that LADA is associated with a significant co-occurrence of autoimmune thyroid disease (as is T1DM). Thus TH issues are constantly lurking in the backgrounds of both T1DM and T2DM.

Appreciation of the co-occurrence of T1DM and T2DM in the same patient requiring changes in classical diabetes management is a relatively new concept. Also, it remains somewhat controversial and is considered cutting edge material. The issues will be addressed and integrated into management protocols in the years ahead. In consideration of the reality regarding this cutting edge material with reference to the present disclosure, the following is stated: the present disclosure claims novelty in regard to its' methods and formulations with applications to T1DM in the same manner as this novelty is claimed in regard to applications to T2DM.

Environmental risk factors for T2DM are reasonably well understood. It is generally accepted that chronic excessive caloric intake is a prerequisite for the development of T2DM. The general lack of regular rigorous exercise in these patients is also a significant contributor. Genetic risk factors, which play a very significant role, are less well understood. Many gene polymorphisms have been identified as being associated with T2DM. A given genetic risk factor may not have the capacity to generate the development of T2DM on its own and the production of the disease may require two or more such genetic risk factors. An example of the requirement for the co-occurrence of two genetic risk factors for the development of T2DM is the co-occurrence of the Thr92Ala polymorphism of the D2 enzyme with the peroxisome proliferator-activated receptor gamma-2 (PPARG2) Pro12Ala polymorphism. This association was initially found to be present in patients with the metabolic syndrome who did not have T2DM {8}. It was subsequently found to be present in patients with T2DM {9}. The Thr92Ala polymorphism is believed to be responsible for 15-20% of patients with T2DM and it has been shown to cause ER stress {10}. However many with this polymorphism do not have T2DM, indicating inconsistency as a sole etiologic cause. Large cohort studies suggest that unless other relevant polymorphisms are present in the same patient the Thr92Ala D2 polymorphism is generally benign.

The teleologic significance of the human organisms' priority to prevent unwanted TH activation has been referenced above. There is perhaps no more dramatic evidence for this than the fact that the chief inactivator of TH, D3, has a half-life of 12 hours, while the chief activator of TH, D2, has a half-life of 40 minutes. D2 is a delicate and vulnerable enzyme primarily because of its short half-life, which can be further shortened under certain conditions. To quote Gereben {1}: “D2 is considered the critical homeostatic T₃-generating deiodinase due to its substantial physiological plasticity. A number of transcriptional and posttranscriptional mechanisms have evolved to ensure limited expression and tight control of D2 protein levels, which is critical for its homeostatic function. D2 activity/mRNA ratios are variable, indicating that there is significant posttranslational regulation of D2 expression. In fact, the decisive biochemical property that characterizes the homeostatic behavior of D2 is its short half-life (˜40 min), which can be further reduced by exposure to physiological concentrations of its substrate, T₄, and in experimental situations, rT₃ or even high concentrations of T₃. This down-regulation of D2 activity by substrate is a rapid and potent regulatory feedback loop that efficiently controls T₃ production and intracellular T₃ concentration based on availability of T₄”.

Metformin {11} is a member of the class of drugs known as the BGs. At this time it is the only member of this class in use. Metformin is on the World Health Organizations's list of essential medicines. Metformin ameliorates insulin resistance by multiple mechanisms. Metformin reduces hepatic gluconeogenesis, which is increased in IR as well as the metabolic syndrome and T2DM. Metformin also increases peripheral insulin sensitivity, enhances peripheral glucose uptake and enhances fatty acid oxidation in the mitochondria. Metformin also decreases absorption of glucose from the gut.

The TZDs {12} act by activating peroxisome proliferator-activated receptors (PPARs), specifically PPAR-gamma (PPARG). Presently the only TZD approved for use in the United States is pioglitazone. The TZDs decrease insulin resistance in adipose tissue, muscle and liver. PPARG agonists bind to DNA at the retinoid x receptor, increasing transcription of certain genes and decreasing transcription of others. An important effect of the TZDs is to increase the storage capacity for free fatty acids in adipose tissue. PPARG receptor agonists and TH receptor agonists have overlapping metabolic effects and regulate a similar subset of genes involved in lipid homeostasis. Crosstalk has been demonstrated between downstream TH and PPARG signaling pathways {13}. This suggests an interdependency or a synergism between TH and TZDs. It may also imply that cellular euthyroidism is a prerequisite for the optimal function of TZD's.

Although contraindications and adverse effects of BGs and TZDs are beyond the scope here, the following is worthy of note: combining one or both of these two drug classes with T3 may protect patients from adverse effects such as lactic acidosis (seen with metformin due to its' effect on inhibition of hepatic gluconeogenesis) and cardiac complications (myocardial infarction and congestive heart failure seen with TZDs, more so with rosiglitazone). This potential for these additional benefits of the formulations of the present disclosure has not been studied.

Adiponectin is a protein hormone and adipokine produced primarily in adipose tissue. Metabolic effects of adiponectin include decreased gluconeogenesis, increased cellular glucose uptake, beta oxidation facilitation, triglyceride clearance, endothelial protection, enhanced insulin sensitivity and weight loss {14}. Adiponectin levels are decreased in both the preclinical and clinical stages of the metabolic syndrome and T2DM {14}. TH upregulates adiponectin production. Hyperthyroid states are associated with elevated adiponectin levels {15} while hypothyroid states are associated with lowered adiponectin levels {15}. Further, TZD treatment is known to result in an elevation of adiponectin levels {14}. Part of the synergistic effect expected from the combination of TH and a TZD is that the elevation in adiponectin levels resulting from combined T3 and TZD administration will exceed the adiponectin elevation provided by either T3 or TZD administered alone, one without the other. This valuable synergistic effect would be expected even at T3 doses in the range of 2-10 mcg/24 hours (which is in the lower half of the T3 dosage range declared in the present disclosure).

FIG. 1 is a diagram descriptive of the setebaiD-Theory. It begins with oxidative stress 1 and ends with beta cell failure 11. Mitochondrial stress 2 and ER stress 3 exist in a vicious cycle 4 in T2DM. The ER stress 3 triggers the UPR 5. The UPR 5 is a natural cellular stress response related to and triggered by ER stress. It is believed conserved in all mammalian species. The UPR 5 aims at restoring normal function to the cell by halting protein translation, degrading misfolded proteins and activating signaling pathways involved in normal protein folding. In the event that these objectives are not achieved within a certain time frame, the UPR 5 shifts its goal to apoptosis by promoting cell death. Because ER stress 3 and mitochondrial stress 2 critically and adversely impact beta cell function, these also result in beta cell stress 6. The UPR triggered in the ER results in protein synthesis failure 7. This in turn results in a failure to maintain vital cell functions, including insulin receptor maintenance and the sustaining of second messenger functions. The insulin receptor is degraded 8 and second messenger functions are degraded 9. This results in an insensitivity to insulin at the cell membrane, known as insulin resistance 10. The beta cell is stimulated to produce more insulin, which results in additional beta cell stress 6. This eventually leads to beta cell failure 11 which is an indication for treatment with exogenous insulin.

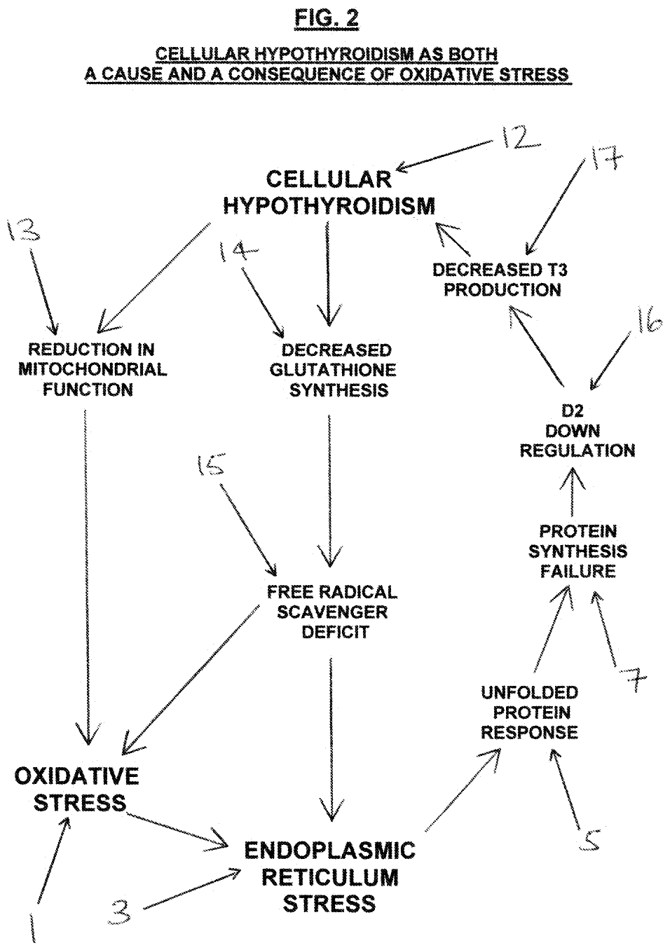

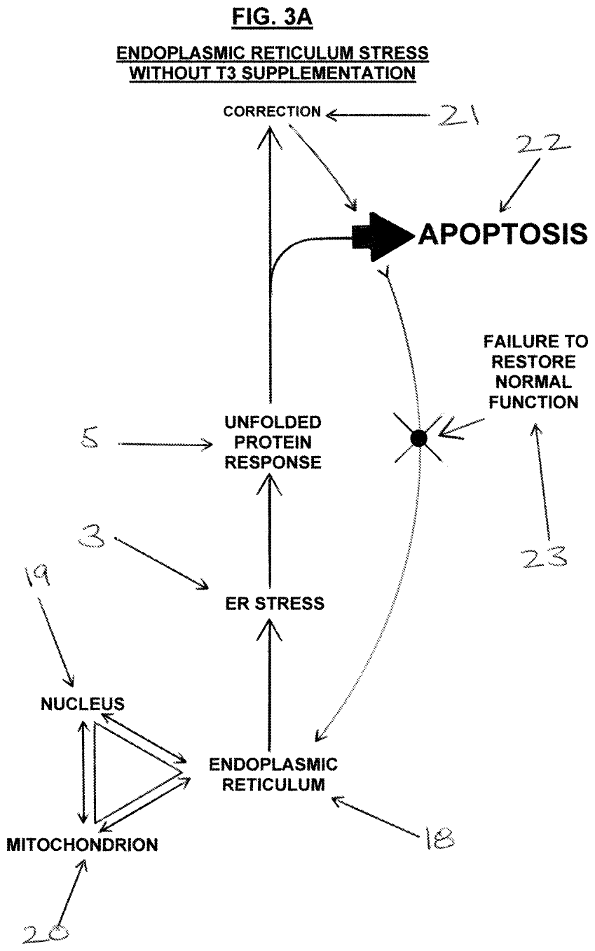

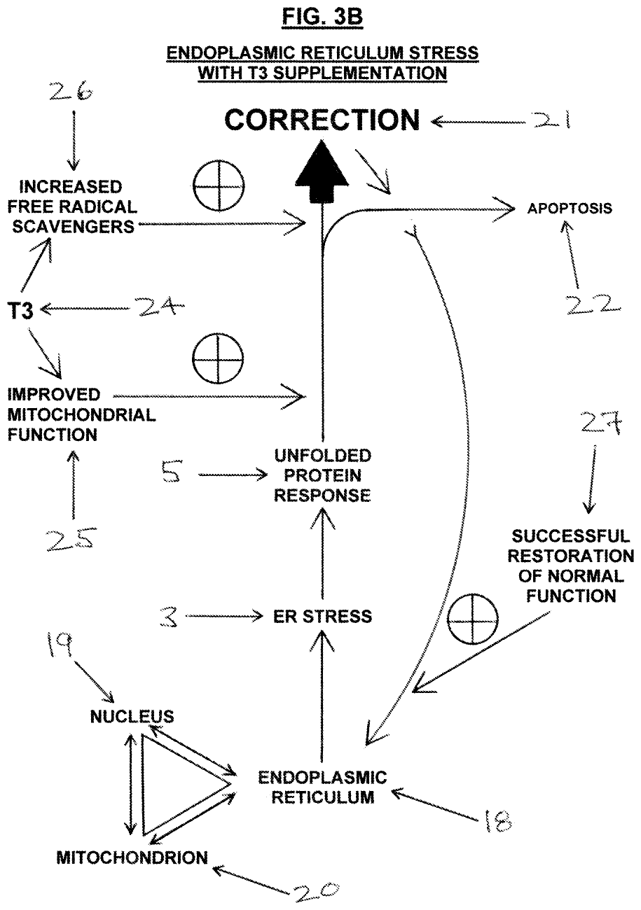

FIG. 2 is a diagram showing cellular hypothyroidism as it relates to the art of the present disclosure. Cellular hypothyroidism is both a cause and a consequence of OS. Cellular hypothyroidism 12, from whatever antecedent mechanism, leads to qualitative and quantitative reduction in mitochondrial function 13. The synthesis of the bodys' chief antioxidant, glutathione (GSH), is decreased 14. This occurs because (i): GSH synthetic enzymes are downregulated in the face of cellular hypothyroidism; (ii): ER stress impairs synthesis of the GSH synthetic enzymes. This reduction in GSH results in a deficit in the number of free radicals scavengers 15. This leads to oxidative stress 1. ER stress 3 occurs because the ER is dependent on normal free radical scavenger function for protein synthesis. The UPR 5 is triggered in the ER. Protein synthesis failure occurs 7. D2 undergoes post-translational downregulation 16 leading to a reduction in the cells' ability to convert T4 to T3. This decrease in T3 production 17 aggravates the cellular hypothyroidism 12, or produces cellular hypothyroidism 12 if it was not present before. FIG. 3A is a diagram showing ER stress as found in conditions of IR (exemplified in the instant case by the metabolic syndrome and type 2 diabetes) without T3 supplementation. The ER 18 is shown as well as the interplay between the ER 18, the nucleus 19 and the mitochondrion 20. One of the more important of these derangements impacting FIG. 3A is the disruption of the activity of D2 which is resident in the ER. The reason that this is important is that T3 is the most potent physiologic regulator of mitochondrial function, both qualitatively and quantitatively. The reduction of D2 activity eliminates a key remedy for the precipitating oxidative stress and, at the same time, aggravates the oxidative stress, as demonstrated in FIG. 2. The ER stress 3 triggers the UPR 5. The UPR 5 aims at restoring normal function to the cell by halting protein translation, degrading misfolded proteins and activating signaling pathways involved in normal protein folding. In the event that these objectives are not achieved within a certain time frame, the UPR 5 shifts its goal to apoptosis by promoting cell death. Under circumstances of cellular hypothyroidism, the UPR 5 lacks the resources to correct the situation. The correction 21 is minimal. Apoptosis 22 is the main outcome. The minimal correction 21 results in a failure to restore normal function 23 to the ER 18. The circumstances characterized in FIG. 3A find cells limping along, retarded by a state which could well be described as involving varying degrees of pre-apoptosis.

FIG. 3B is a diagram showing ER stress 3 when T3 24 is supplemented to compensate for the deficient D2 activity in the ER. The ER 18 is shown as well as the interplay between the ER 18, the nucleus 19 and the mitochondrion 20. The supplemented T3 24 is now able to fulfill its' molecular biologic mandate. In this setting the ER stress 3 triggers the UPR 5 and the cell now has the resources required in order to maximize the required correction 21 in the ER and to minimize apoptosis 22. These resources facilitate improved mitochondrial function 25 and increase the number of free radical scavengers 26. The correction 21 is now able to result in successful restoration of normal function 27 in the ER 18. Some elaboration is needed here or else the above disclosed material will appear overly simplistic. The ER stress without T3 and the restoration of ER function with T3, respectively, are not ‘nothing’ and ‘all’ phenomena. A reasonable example, without limitation or exclusion, is that in FIG. 3A the ER function without T3 would be less than or equal to 30% of normal and in FIG. 3B the ER function with T3 24 would be greater than or equal to 70% of normal. In regard to GSH referenced in FIG. 2 and free radical scavengers referenced in FIG. 3B it is prescient here to highlight the reason why antioxidant therapy has been a universal failure in attempts to correct the OS in T2DM. The reason is that all antioxidants, exogenous and endogenous, must work through the monopolistic broker that is GSH. If the broker is broken, there can be no effective redox market in the human body.

Thus a key pathophysiology, the development of cellular hypothyroidism, is defined here as being responsible for the transitioning of the metabolic syndrome and T2DM from their pre-clinical to their clinical stages. Further this pathophysiology is remedied by the art of the present disclosure.

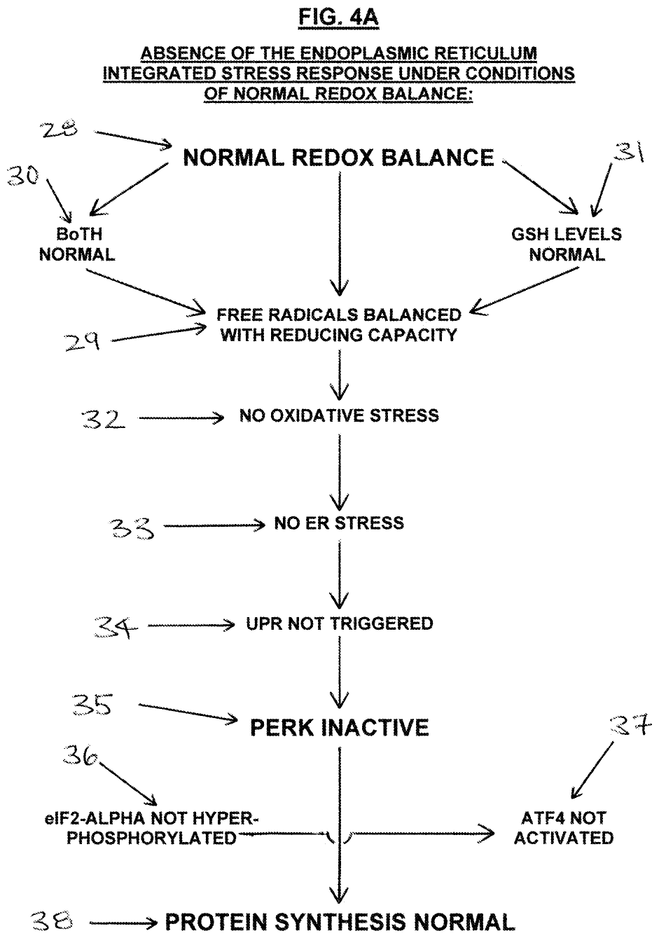

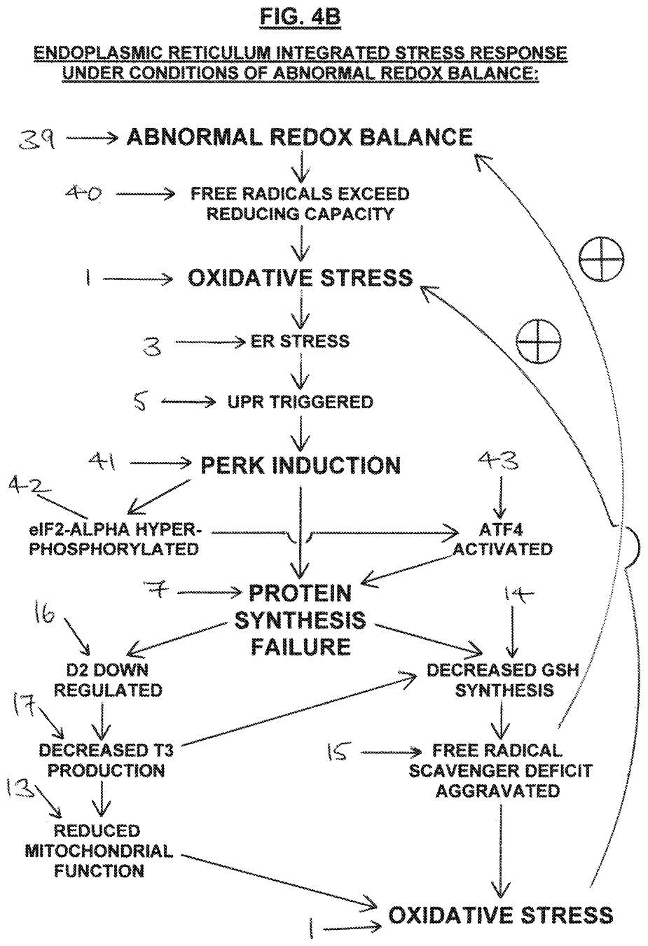

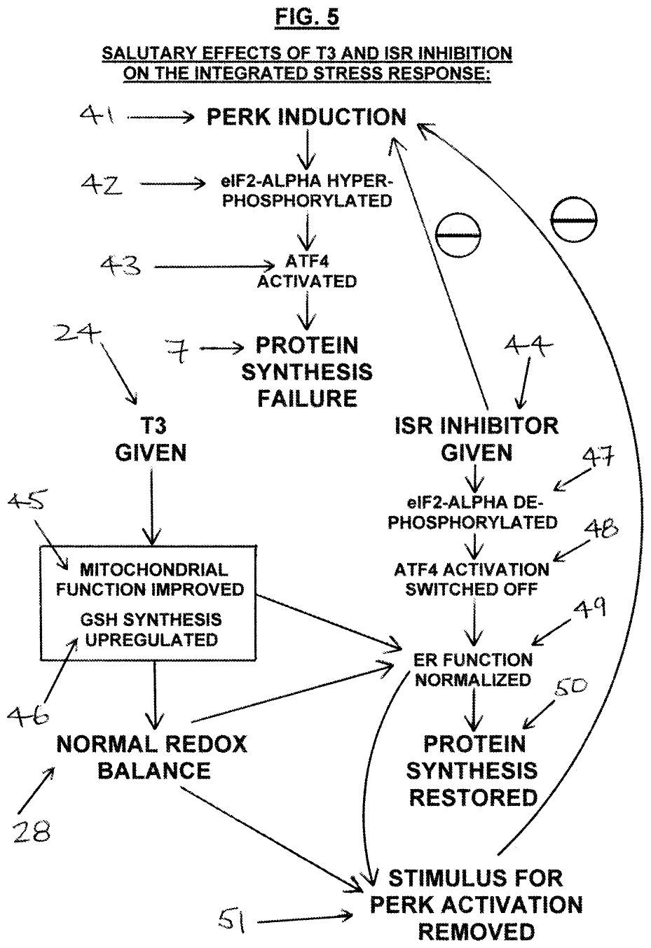

FIG. 4A shows the ER absent the ISR, which is part of the UPR, under conditions of normal redox balance. FIG. 4A is shown here in order to facilitate comparison with, and understanding of, FIG. 4B and FIG. 5. When normal redox balance 28 exists in the cell, free radicals are balanced with reducing capacity 29. Prerequisites for this state are a normal BoTH 30 and normal GSH levels 31. Consequently there is no oxidative stress 32 and thus no ER stress 33. The UPR is not triggered 34. The PERK pathway remains inactive 35. Consequently eIF2a in not hyper-phosphorylated 36 and ATF4 is not activated 37. Protein synthesis is normal 38.

FIG. 4B shows the activated ER ISR under conditions of abnormal redox balance. In the setting of abnormal redox balance 39 free radicals exceed reducing capacity 40. Oxidative stress 1 is present. The ER experiences ER stress 3 and the UPR 5 is triggered. PERK pathway induction 41 occurs. The PERK kinase enzymes produce eIF2a hyper-phosphorylation 42 which leads to activation of ATF4 43. This results in protein synthesis failure 7. D2 is downregulated 16 and there is decreased T3 production 17. GSH synthesis is decreased 14, due to both protein synthesis failure 7 and downregulation of GSH synthetic enzymes in the presence of a paucity of T3, which normally upregulates them. The lower GSH levels aggravate the free radical scavenger deficit 15. Paucity of T3 also reduces mitochondrial function 13, qualitatively and quantitatively. Thus the knock-on effects of protein synthesis failure 7 all contribute to an increase in the oxidative stress 1 which becomes a positive feedback loop.

FIG. 5 shows the salutary effects of T3 administration and the administration of an ISR inhibitor on the pathophysiology of FIG. 4B. FIG. 5 again shows PERK induction 41 leading, via hyper-phosphorylated eIF2a 42 and ATF4 activation 43, to protein synthesis failure 7. The T3 24 given produces an improvement in mitochondrial function 45 and upregulation of GSH synthesis 46, both of which restore normal redox balance 28. The ISR inhibitor 44 given leads to de-phosphorylation of eIF2a 47 and this switches off ATF4 activation 48. The restoration of redox balance 28 and the switched off ATF4 activation 48 result in ER function being normalized 49. Protein synthesis is restored 50 and the stimulus for PERK activation is removed 51. The existence of a synergism between T3 and an ISR inhibitor in combating IR is proposed and explained: as the sole therapeutic agent (without T3) used to remedy the instant pathophysiology described in FIG. 4B, the use of ISR inhibition is directed to blocking a natural reparative process where the outcome is corrective repair or apoptosis. ISR inhibition does nothing to eliminate the factors which resulted in the cell finding itself in a dire situation in the first place. Use of ISR inhibition alone is simplistic and its' promise of repair to the ER comes with the risk that some other element in the cell will break down. The co-administration of T3 with ISR inhibition, for reasons relating to the narrative on FIG. 5, actually fixes something (the redox balance, explained in the narrative on FIG. 5) rather than just blocking a reparative pathway which appears to be getting in the way.

In another embodiment of the present disclosure ERT3 is combined in a polypharmaceutical with other drugs which antagonize insulin resistance so that IR can be addressed by a multi-targeted and multi-drug approach. ERT3 is combined with a BG and/or a TZD and/or an ISR inhibitor. To this end protocols are presented below which enable the transitioning of patients with the metabolic syndrome and T2DM to treatment regimens of the present disclosure and which involve cautious multi-stage approaches. These are necessary in order to ensure that the drugs are well tolerated by the patient, to attain the maximum dosing permutation in the polypharmaceutical, as appropriate for a given patient, and to prevent hypoglycemic episodes.

Prior to beginning the regimens, patients taking exogenous insulin are changed from shorter acting insulin preparations to a longer acting insulin, such as insulin glargine, where the daily dose is given once every 24 hours or some version of the split daily dose is given every 12 hours. The reason for this is as follows: even when this regimen is administered cautiously, there is a small risk of hypoglycemia. Hypoglycemic episodes are milder and easier to treat with a long acting insulin than they are with a short acting insulin. While the medication regimen should be administered in a slow cautious uptitration, the lowering of the daily insulin dose will need to be more aggressive, particularly at the initiation of the protocol. While examples of drugs and doses are given below, this is done for illustrative purposes and not for the purpose of limitation or exclusion. The first stage of this example involves low doses of all drugs in the polypharmaceutical which is dosed every 12 hours: The drugs used in this example are ERT3, T4, metformin and pioglitazone. T4 is added to T3 here for reasons explained below. Examples of low twelve hourly doses in this first stage would be: T3=2 mcg; T4=10 mcg; metformin=650 mg; pioglitazone=10 mg. Once the patient is tolerating this regimen the second stage begins. In the second stage the ERT3 and T4 doses are left at the levels used in the first stage, but the metformin and piolitazone doses are increased as tolerated, even to the point of their maximum doses. In the case of metformin this is approximately 1,250 mg/12 hours and in the case of pioglitazone this is approximately 22.5 mg/12 hours. Once dose titration of the metformin and pioglitazone has been accomplished, the third stage begins. In the third stage the ERT3 dose is uptitrated while the doses of T4, metformin and pioglitazone remain unchanged. The ERT3 dose is uptitrated from 2-3 mcg/12 hours; then from 3-4; then from 4-5; then from 5-6; and so on. This ERT3 uptitration should be performed over a number of weeks. The majority of patients are expected to require daily ERT3 doses of 12 mcg or less (6 mcg/12 hours). The sole purpose of the T4 treatment is for maintenance of a normal plasma TSH level for reasons described below. If the plasma TSH level rises on this polypharmaceutical drug regimen, then the daily T4 dose should be increased from 20 to 40 mcg/24 hours or from 40-60 mcg/24 hours. This should be done before increasing the ERT3 dose. Conversely, if the plasma TSH level falls on this polypharmaceutical regimen, then the T4 dose should be decreased accordingly. This should be done before decreasing the ERT3 dose. There will be occasional exceptional patients who experience TSH suppression which is accomplished by low to intermediate dose ERT3, or who appear to oversuppress their TSH, whether this is in response to T4 or to T3. These exceptions are beyond the scope here and management decisions are left in the hands of a competent treating physician. The above referenced recommendations for management of TSH aberrations and caution about the need to lower insulin dose apply to all transition protocols. These apply regardless of whether ERT3/T4 is being used alone or as a component of the polypharmaceutical as exemplified above.

Below are described method protocols for transitioning patients with IR to the treatment art of the instant disclosure:

Protocol A involves the use of ERT3 (with or without T4) alone in patients receiving exogenous insulin as the sole antidiabetic treatment.

Protocol B involves the use of ERT3 (with or without T4), a BG and/or a TZD in patients receiving exogenous insulin as the sole antidiabetic treatment.

Protocol C involves elements of protocols A and B in patients who are not receiving exogenous insulin.

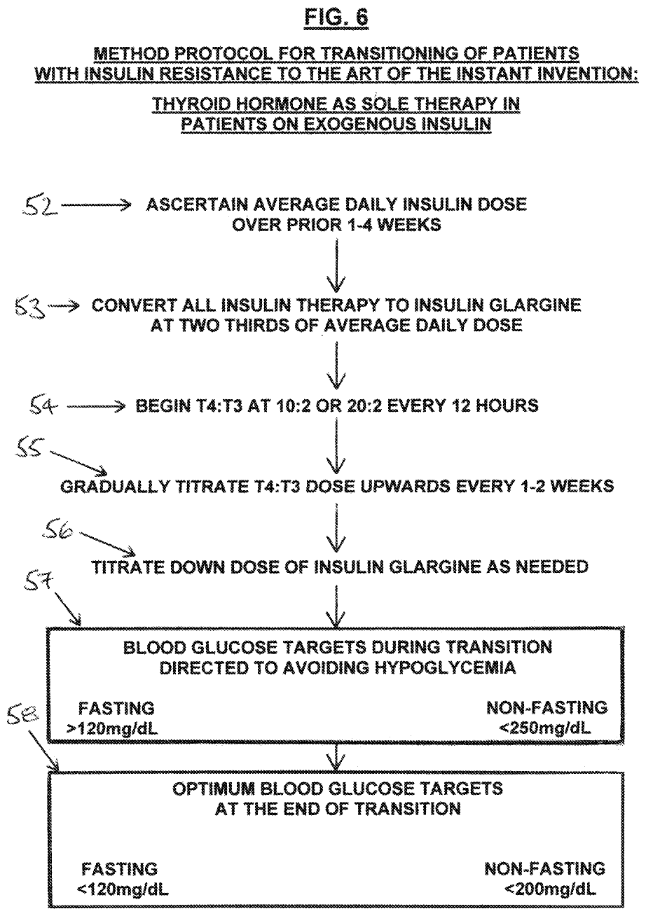

FIG. 6 references Protocol A: The average daily insulin dose used during the prior 1-4 weeks is ascertained 52 (using the total number of units of insulin from all forms of insulin used). The insulin administered is exclusively insulin glargine (or equivalent insulin) commencing at two thirds the calculated average daily dose 53. The T4:T3 combination therapy is begun at a dose of 10:2 or 20:2 administered every 12 hours 54. The TH dose is gradually titrated upwards every 1-2 weeks 55. The insulin glargine dose is titrated down as needed 56. Blood glucose targets during transition 57 are prioritized to prevent hypoglycemia as shown. Blood glucose targets at the end of the transition 58 are prioritized to provide optimum glycemic control assuming that there is no contraindication based on disease comorbidities. Alternatively blood glucose goals are structured according to consensus recommendations for a given patient. A variant of protocol A is activated when, prior to initiation of the T3, the patient is taking oral antidiabetic agents in addition to insulin. While it is considered generally safe to leave the doses of oral agents unchanged and to proceed with the protocol as disclosed, these decisions are left to be made by a competent treating physician.

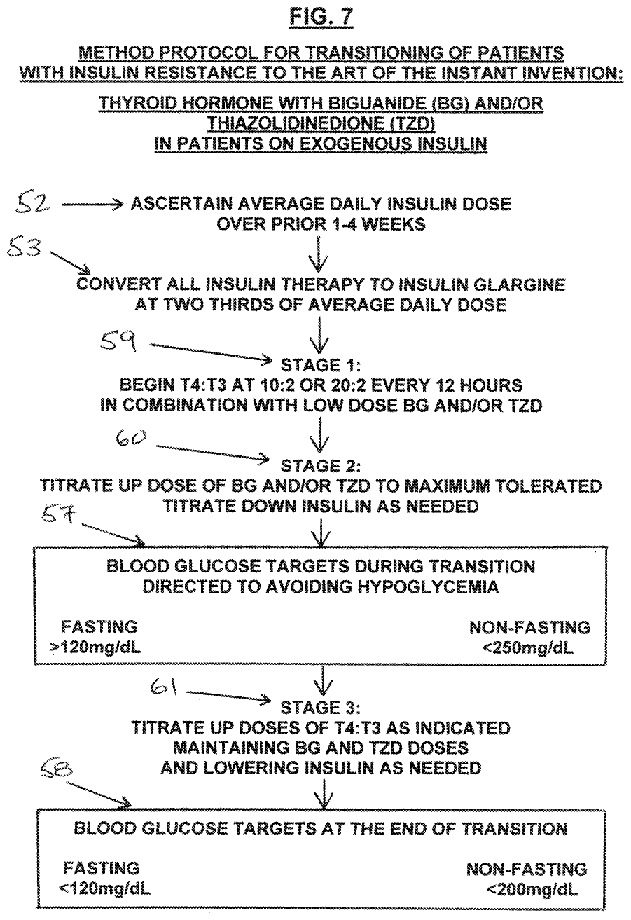

FIG. 7 references protocol B: The average daily insulin dose is ascertained 52 in the same manner as in FIG. 6. The patients' insulin is converted to insulin glargine, or equivalent, commencing at a two thirds the calculated average daily dose 53. During stage 1 59 the T4/T3 is begun at a dose of 10:2 or 20:2 together with low dose BG and/or low dose TZD. In stage 2 60 the BG and/or TZD are titrated up to the maximum dose recommended or tolerated and the insulin dose is titrated down as needed, while the dose of the T4:T3 combination is left unchanged. Blood glucose targets during transition 57 are prioritized to avoid hypoglycemia. In stage 3 61 the T4/T3 combination is titrated up to optimum levels, BG and TZD doses are maintained and insulin is lowered as needed. Blood glucose targets at the end of transition 58 are optimal unless a contraindication exists. A variant of protocol B is activated when, prior to initiation of the transition to the polypharmaceutical, the patient is taking oral antidiabetic agents, which may or may not include a BG and/or a TZD, in addition to insulin. The knowledge required to in order to configure the required protocol here is known to those skilled in the art and these decisions are left in the hands of a competent treating physician.

Protocol C: Protocol C is not accompanied by a diagram. It is used in patients who are not receiving exogenous insulin. Appropriate elements of protocols A and B are used. The main difference with protocols A and B is as follows: prior to stage 1 the doses of all antidiabetic drugs (specifically insulin secretagogues and other drugs whose action is to enhance insulin secretion or activity) are reduced by 50%. Thereafter the other elements of protocols A and B are implemented as appropriate with gradual uptitration of the TH pharmaceutical dosage and down titration or discontinuation of the antidiabetic drugs.

It will be apparent that numerous similar protocols are possible, because the requirements for the specifics of the protocol are that it be tailored to the patients' pre-existing drug regimen and to the intended glycemic control goal for the patient in question. The guiding principles of all of these protocols, specifics disclosed or undisclosed, are as follows: (i): Avoidance of hypoglycemia is the priority; (ii): Exclusive use of insulin glargine or equivalent where insulin is required; (iii): Avoidance of medication adverse effects; (iv): Gradual titration (up or down) of medication; (v): Ultimate glycemic control and HbA1C targets are determined for a given patient by expert panel recommendations; (vi): Ideally and ultimately, cessation of all exogenous insulin is an important goal if possible; (vii): Lifestyle recommendations including diet and exercise should be adhered to.

Thyroid hormone is protean to all vertebrates and its' metabolic effects are ubiquitous. Consequently, it should not be surprising that there may be other benefits to using TH in the metabolic syndrome and T2DM which are related or unrelated to IR or gylycemic control. Related to IR the beneficial effect of TH is particularly important in adipose tissue, in the pancreatic beta cell, in liver and in muscle, with benefits to cellular organelles, in particular the ER and the mitochondrion. TH is vital to adipocyte physiology, including differentiation of the adipocyte cell line from the pluripotent germ cell. Maintenance of adiponectin levels by TH has been referenced above. Another important function of TH is the facilitation of beta oxidation (breakdown of free fatty acids for use as an energy source) in the mitochondria. Beta oxidation is reduced in IR. Unrelated to IR, TH is a potent and natural mood stabilizer/antidepressant and its' effects on the human brain enhance the impetus to pursue physical activity. Effects on the gut optimize drug absorption.

There is a high likelihood that a synergism exists between all four drug classes of the instant disclosure (T3; BGs; TZDs; ISR inhibitors) in regard to the antagonism of insulin resistance. In particular, there is expected synergism between T3 and TZD's and there is expected synergism between T3 and ISR inhibitors. The expected synergism between T3 and TZD's in relation to downstream crosstalk signaling has been referenced above. The expected benefit of synergism between ERT3, a BG and a TZD is discussed below. Expected synergism between T3 and ISR inhibition warrants explanation. On its' own, the use of ISR inhibition is directed to blocking a natural reparative process where the outcome is corrective repair or apoptosis. ISR inhibition does nothing to eliminate the factors which resulted in the cell finding itself in a dire situation in the first place. Use of an ISR inhibitor alone is simplistic and its' promise of repair comes with the risk that something else in the cell will break down. The co-administration of T3 together with an ISR inhibitor, for reasons relating to the narrative on FIG. 3B and FIG. 5, actually fixes something (the redox balance) rather than just blocking a reparative pathway which appears to be getting in the way. Because other conditions have pathophysiology involving IR and ER stress, this discourse on synergism is also relevant to remedies for other conditions associated with IR and ER stress, in particular to Alzheimers disease, atherosclerotic vascular disease and pre-eclampsia.

A method for ameliorating IR is provided and, in addition, descriptions for the enablement of compositions for formulation of therapeutic agents, directed towards the amelioration of IR, are provided. Furthermore, a method for the application of a novel synergism of therapeutic agents to combat IR is presented. Enablement protocols are also disclosed to aid in transitioning patients to the art of the present disclosure. The method for ameliorating IR comprises formulation and administration of an extended release active ingredient, wherein the active ingredient may be T3, being L-triidothyronine, liothyronine, liothyronine sodium, or similar formulations or compounds, which may safely antagonize IR in patients with the metabolic syndrome and T2DM. In another embodiment ERT3 is combined in a polypharmaceutical with a BG and/or a TZD and/or an ISR inhibitor to achieve synergism in antagonizing IR.

A method for treating IR with T3 being L-triiodothyronine, liothyronine, liothyronine sodium, or similar formulations in an extended release formulation allows patients to be treated for IR in a safe manner. Extended release caplets or tablets or other suitable vehicle for administration, being via oral, injectable, or other suitable route of administration to a human patient, not limited to a tablet, capsule, gelcap, a powder dispensed in a beverage, orally disintegrating tablet, a vial, ampule, or other container of liquid such as a solution or suspension, a lozenge, lollipop, gum, inhalers, aerosols, injectables, creams, gels, lotions, ointments, balms, eye drops, suppositories, and patches, with the minimum effective T3 dose tailored to the individual patient. For instance, said dosage of ERT3 being at least 2 mcg, or at least 4 mcg, or at least 6 mcg, or at least 8 mcg, or at least 10 mcg, or 12 mcg, or at least 14 mcg, or at least 16 mcg, or at least 18 mcg per 24 hours, overcomes concerns regarding immediate release formulation of T3, described below, resulting in lower plasma concentration levels. Alternately a drug dispensing device may be implanted either sub-dermally or otherwise and configured to release T3 in a slow manner. The post absorptive blood levels of this extended release T3 could more closely resemble a steady state or constant level of T3 in the blood rather than a high spike in post-absorptive blood levels of the immediate release formulation, thereby avoiding supra-physiologic or high plasma concentration of T3 levels in the blood. This tailoring to the individual patient may be achieved by the treating physician making judgments based on the patients' symptoms and signs, results of thyroid function tests (T3, T4, and TSH), blood glucose monitoring and HbA1C monitoring.

Because TH modulates the sympathetic nervous system, excessive plasma levels of T3 can lead to cardiac complications which include cardiac hypertrophy, arrhythmias and high output heart failure. Even in the absence of sustained chronic T3 excess, immediate release T3, with its' supraphysiologic post-absorptive plasma levels, may produce cardiac arrhythmias, chiefly supraventricular. Therefore, immediate release T3 is not suitable, especially for older patients. Absorption of T3 (L-triidothryonine or liothyronine) is 90% with peak levels reached one to two hours following ingestion. Plasma concentration, or amount of drug in circulation, may rise by 250% to 600%. T3 has a half-life in humans of 19 hours {16}. Single dose, immediate release T3 ingestion may place a patient at risk for cardiac arrhythmias, chiefly but not limited to supra-ventricular arrhythmias, and potentially other adverse effects. Consequently, the American Geriatric Society has designated DT (containing immediate release T3) as fitting the Beers Criteria, indicating a need for avoidance, or use with caution, in older adults.

The methods and therapeutic compositions described herein are for the purpose of ameliorating IR by the administration of L-tri-iodothyronine to a human patient via an extended release formulation. Further a combination of ERT3 and T4 may be administered for reasons explained below. In addition a method for the formulation of a therapeutic agent for treating IR is presented. A state of cellular hypothyroidism, that being a surfeit of intracellular 3,5,3′-L-tri-iodothyronine, is proposed here as being the major causative factor in the development of clinically evident IR, that being present in overt clinical syndromes including, but not limited to, the metabolic syndrome and T2DM, when the diseases are no longer subclinical. Cellular hypothyroidism occurs because OS results in ER stress which in turn compromises the activity of the protein, enzyme and ER resident that is D2, the main activator of TH. Cellular hypothyroidism is usually not the primary cause of IR in the earliest stages of these diseases, although rarely it may be. The diseases more commonly have primary genetic and/or environmental origins which are not TH related.

Extended release formulations and/or controlled and/or delayed-release dosage forms (collectively now referred to as ‘modified release’) have been used since the 1960s to enhance performance and increase patient compliance while also potentially minimizing unwanted side effects. The dosage forms may comprise those configured to release the active ingredient over a four-hour period, or over an eight-hour period, or a twelve, twenty-four hour, thirty-six hour, or even forty-eight hour period. Alternately the release may be a delayed release in that the active ingredient doesn't reach significant levels in the blood until about one to four hours after dosing with release over the next twenty-four to twenty-six hours or more including thirty-six or forty-eight hours. Total twenty-four hour or daily intake of the active ingredient T3 may be at least 1 μg, or 2 μg of active ingredient, or at least 4 μg, or at least 6 μg, or at least 8 μg, or at least 10 μg, or at least 12 μg, or at least 14 μg, or at least 16 μg or at least 18 ug. In other embodiments, the unit dosage form may comprise one or more extended-release dosage forms which are configured to release the active ingredient over a period of days such as in the case of the internal device as described above. Matrix type extended release systems or diffusion-controlling membranes, or other extended release technologies may be employed. Non-active inert ingredients for drug delivery may be included in formulations. Matrix type systems may be based on hydrophilic polymers wherein the drugs and excipients, being non-active inert ingredients, are mixed with polymer such as hydroxypropyl methylcellulose (HPMC) and hydroxypropyl cellulose (HPC) and then formed as a tablet by conventional compression. Water diffuses into the tablet, swells the polymer and dissolves the drug or active ingredient, whereupon the drug may diffuse out being released into the body. This type of technology is open to mechanical stress from food substances which may lead to increased release rate and a higher risk of dose-dumping. These systems also require a large amount of excipient and drug loading is comparatively low. Use of diffusion-controlling membranes is another method of obtaining extended or controlled release of active ingredients. With this technology, a core that may be pure active ingredient, or mixture of active ingredient and excipient(s), is coated with a permeable polymeric membrane. Water diffuses through the membrane and dissolves the drug which then diffuses out through the membrane at a rate determined by the porosity and thickness of the membrane. Membrane polymers may be those such as ethylcellulose.

Oral extended release or controlled release formulations may be of several types. Matrix type extended release systems or diffusion-controlling membranes, or other extended release technologies may be employed. Non-active inert ingredients, which may also be excipients for drug delivery and/or needed for formulation may be included. In another embodiment, T3 may be formulated together with T4, or levothyroxine or L-thyroxine, to maintain T4 levels, as explained below. Levothyroxine is a synthetic thyroid hormone that may be available under the names Levothroid, Levovyxl, Levo-T, Synthroid, Tirosint, and Unithroid. T3 or triidothyronine and/or similar compounds may be suitable for administration in extended release, also termed controlled release, dose formulations. Extended release formulations may be presented in vehicles not limited to a tablet, capsule, gelcap, a powder dispensed in a beverage, orally disintegrating tablet, a vial, ampule, or other container of liquid such as a solution or suspension, a lozenge, lollipop, gum, inhalers, aerosols, injectables, creams, gels, lotions, ointments, balms, eye drops, suppositories, and patches. Alternately an internal device may be implanted in the patient and release T3 over time. Active ingredient concentration or dose amount may be described as appropriate depending on weight and/or size of the patient. The dosing regimen that follows is given here is for example purposes and not for limitation or exclusion. Twelve hourly dosing of ERT3 may be greater than or equal to 1 mcg and less than or equal to 10 mcg, or a different dose and/or dosing interval may be used. It is anticipated that many patients with IR in association with the metabolic syndrome and T2DM will derive substantial benefit from T3 doses as low as 2-6 mcg/24 hours, with a lowering of IR by said T3 dosage range.

A subset of patients taking T3 monotherapy (T3 without T4) will show thyroid function tests which demonstrate an apparently spurious rise in TSH. This occurs because the levels of plasma T3 generated in these patients are insufficient to result in central negative feedback inhibition/suppression of TSH. This central negative feedback inhibition/suppression of TSH is primarily a T4 mediated phenomenon, mediated by T3 only at higher blood levels in certain patients. The origin of the apparently spurious rise in TSH is explained: while the therapeutic T3 plasma level in this subset of patients is too low for central negative feedback inhibition/suppression of TSH, it is not too low to produce negative feedback directly to the thyroid gland. This effect reduces production and secretion of T4 by the thyroid gland. As a consequence, the plasma level of T4 falls, reducing the central feedback inhibition/suppression of T4 on the central apparatus and thus the TSH rises. This phenomenon results in an elevated TSH, suggesting a hypothyroid state, when in fact the patient is euthyroid by virtue of the T3 treatment. Therefore, in another embodiment, ERT3 may be formulated together with T4, or the two may be given in separate formulations at the same time, thereby maintaining T4 levels with a sufficiency such that central negative feedback inhibition is maintained and a normal TSH is preserved. Further, the option of variable ratios of T3 and T4 in the formulation allows for customized patient formulation as described. In regard to the T4 in the formulation it should be noted that due to the low potency of T4 (it is the pre-hormone) and because of its' relatively long half life of 5-7 days, there is no advantage to be gained by providing T4 in the formulation of the present disclosure in an extended release format. Whether the T4 formulated for the present disclosure is immediate release or extended release is merely a matter of convenience for the pharmacist. Thus the formulation of T4 in the present disclosure allows for either immediate release or extended release T4. Any new art coming after the fact and attempting to circumvent the intellectual property derived from the present disclosure by introducing claims language for the addition of extended release T4 to the formulation should be considered specious.

Thus, it is appreciated that the optimum pharmaceutical in the instant case may be an extended release formulation of T3, optionally combined with T4 with variable T4/T3 ratios allowing for customized patient formulation. Multiple dosage permutations are another objective of the instant invention. In the embodiment combining T4 and T3, The total daily dose of T4 may be 20, 40 or 60 mcg or a different dose. The total daily dose of T3 may be between 2 mcg and 20 mcg. It would be most unusual for a T3 dose in excess of 20 mcg/24 hours to be required. Further, in this setting, the risks of using this dose of >20 mcg would likely outweight the benefits. Even numbered 24 hour doses of ERT3 and T4 are deliberately structured as being even-numbered so that they can easily be divided in two in order to structure the 12 hourly dose. Thus with the foregoing schedule (T4=20, 40 or 60; T3=2−20), the 12 hourly dose would be as low as T4:T3=10:1 and as high as 30:10, with all possible permutations in between.

In other embodiments a method wherein the concentration of L-tri-iodothyronine agonist at the thyroid hormone receptors in the pancreas, liver and the peripheral tissues is increased is described. Further a method comprising maintaining a patient on the lowest effective therapeutic dose is described, where this is defined as the dose whereby, after gradual increase of T3 concentration to the patient, glycemic control is maximized, IR is minimized, exogenous insulin requirements are minimized and the foregoing is achieved in the absence of medication side effects. The method may result in blood levels of T3 more closely approaching steady state blood levels compared with the administration of an immediate release formulation of T3. Further, the method of may be used in patients with TSH levels which are within the normal ranges. The method may also be used in patients with TSH levels which are outside the normal ranges with the following caveat: patients found to have TSH levels below the lower limit of normal will require mandatory specialist consultation prior to being placed on ERT3 in order to determine whether the abnormal laboratory finding is due to subclinical hyperthyroidism, central hypothyroidism or some other cause.

It is anticipated that the optimum dosing interval will be every 12 hours, although other intervals may also be appropriate without departing from the spirit and scope of the invention. The half-life of T3 in humans is 19 hours {16}. With dosing every 12 hours, low steady state plasma levels will be attained while ensuring continuous genomic and non-genomic effects. The 12 hour dosing interval is a good balance between patient compliance and the maintenance of a low steady state plasma level of T3. Twelve hour dosing is also most appropriate for the application of combining ERT3 with a BG and/or a TZD as described.

Attention now turns to defining embodiments of the present disclosure, the composition of the pharmaceutical and the optimum manner in which it is administered to patients in order to ameliorate the metabolic syndrome, T2DM and potentially other conditions, stated or unstated, associated with IR.

Two example formulations, with example dosing intervals, of the present disclosure are shown:
1. Example formulation (1) provides for the addition of the ERT3 formulation (with or without T4) described above to the existing drug regimen of the IR patient with or without initial alteration of said existing regimen. However, in the latter case, subsequent alteration of the antidiabetic regimen may become necessary as the T3 improves glycemic control which then necessitates the reduction of drug doses. Further, in the event that the existing regimen is not modified initially, the initial dose of ERT3 should not exceed 4 mcg/24 hours. The daily dose of the ERT3/T4 combination is divided, with 50% of the daily dose given every 12 hours. The daily dose of ERT3 is expected to be between 2 mcg and 20 mcg although exceptions will occur. The daily dose of T4 is expected to be between 20 mcg and 60 mcg, although exceptions will occur.
2. Example formulation (2) provides for the implementation of the polypharmaceutical method described above consisting of ERT3 (with or without T4) and a BG and/or a TZD and requires the implementation of a protocol for transitioning IR patients to this regimen as described above. The same narrative for ERT3 and T4 referenced above in example formulation (1) applies to example formulation (2). The daily doses of all drugs in this formulation are divided, with 50% of the daily dose of each drug given every 12 hours. The only exception to this 50/50 split is the BG. As an example, without limitation or exclusion, the split may be 40% am and 60% pm or 60% am and 40% pm or the split may be of a different ratio. This flexibility is necessary in order to accommodate unique patient circumstances and prescribing physician preference. The pharmacodynamic reason for this is that the BG is the only component of the polypharmaceutical which has the potential for an hour-to-hour effect on blood sugar. Factors affecting this include the fasting or non-fasting state, exercise and other lifestyle factors. In contrast the T3 and TZD components have a steady state pharmacodynamic effect and do not have a variable hour-to-hour effect on blood glucose.
   In regard to example formulation (2): The BG in the formulation may be formulated for immediate release or extended release. Extended release BG formulation has a preferred status here for reasons relating to maximizing daily dosing and minimizing the number of doses per day (thereby maximizing drug effect and patient compliance). The TZD may be formulated for immediate release or extended release. At the time of writing, no benefit to extended release TZD formulation is known. Unless new art becomes available which suggests a benefit to extended release TZD, the preferred formulation of the TZD in example formulation (2) is immediate release.

Furthermore, the synergism between the components of the polypharmaceutical of example (2) is expected to result in an additional benefit in that this synergism may result in a lower dosage requirement for all drug components in the polypharmaceutical compared with the scenario where each drug component is used as a stand alone monotherapy. An example follows, without limitation or exclusion: a polypharmaceutical containing ERT3 4 mcg, metformin 2,000 mg and pioglitazone 30 mg (24 hour/daily dose) may be equally, or more, effective compared with 24 hour/daily monotherapy with ERT3 8 mcg, metformin 2,500 mg or pioglitazone 45 mg.

Regarding the availability of the specific drugs embodied in the methods and formulations of the present disclosure: T3 is FDA approved and available and is generally formulated for extended release by compounding pharmacies; the BG metformin is available and FDA approved and is available in immediate and extended release formulations; the TZD pioglitazone is FDA approved and available in an immediate release formulation only; in regard to ISR inhibitor drugs none is FDA approved or available. This class of drugs is in its' infancy and current use is limited to research in animals.

It should be noted that the examples described above are provided for purposes of illustration, and are not intended to be limiting. Other formulations, dosing regimens, and devices and/or device configurations may be utilized to carry out the disclosure described herein. It can be envisioned that technology advances in the field may lead to variations of the disclosure.

### REFERENCES

1. Gereben B, et al. 2008: Cellular and Molecular Basis of Deiodinase-Regulated Thyroid Hormone Signaling. Endoc. Rev. December; 29 (7) 898-938.
2. Arroyo e Drigo R, et al. 2011: Endoplasmic Reticulum Stress Decreases Intracellular Thyroid Hormone Activation via an elF2a-Mediated Decrease in Type 2 Deiodinase Synthesis. Molec. Endocrinol. December; 25 (12) 2065-75
3. Gronich N, et al 2015: Hypothyroidism Is a Risk Factor for New-Onset Diabetes: A Cohort Study. Diabetes Care 38; 1657-1664
4. Chang y, Chuang L 2010: The role of oxidative stress in the pathogenesis of type 2 diabetes: from molecular mechanism to clinical implication. Am J Transl Res 2 (3) 316-331
5. Krukowski K, et al 2020: Small molecule cognitive enhancer reverses age-related memory decline in mice. doi.org/10.1101/2020.04.13.039677
6. Dentice M, Salvatore D 2011: Deiodinases: the balance of thyroid hormone: Local impact of thyroid hormone inactivation. J. Endocrinol. 209 (3): 273-282
7. Priya G, Kalra S 2018: A Review of Insulin Resistance in Type 1 Diabetes: Is There a Place for Adjunctive Metformin? Diabetes Ther 9: 349-361
8. Fiorito M et al 2007: Interaction of DIO2 T92A and PPARG2 P12A Polymorphisms in the Modulation of Metabolic Syndrome. Obesity 15 (12) 2889-95
9. Estivalet A, et al 2011: D2 Thr92Ala and PPARG2 Polymorphisms Interact in the Modulation of Insulin Resistance in Type 2 Diabetic Patients. Obesity 19 (4) 825-32
10. Jo S, et al 2019: Type 2 deiodinase polymorphism causes ER stress and hypothyroidism in the brain. J Clin Invest 129 (1); 230-245
11. Zhou J, et al 2018: Metformin: an Old Drug with New Applications. Int J Mol Sci 19 (10) 2863-2878
12. Barnett A H, 2009: Redefining the role of thiazolidinediones in the management of type 2 diabetes. Vasc Health and Risk Man 5; 141-151
13. Lu C, Cheng S 2010: Thyroid hormone receptors regulate adipogenesis and carcinogenesis via crosstalk signaling with peroxisome proliferator-activated receptors. J. Mol. Endocrinol. 44 (3); 143-154
14. Yanai H, Yoshida H 2019: Beneficial Effects of Adiponectin on Glucose and Lipid Metabolism and Atherosclerotic Progression: Mechanisms and Perspectives. Int. J. Mol. Sci. 20; 1190-1214
15. Seifi S, et al 2012: Regulation of adiponectin gene expression in adipose tissue by thyroid hormones. J. Physiol. Biochem. 68; 193-203
16. Walsh M 2006: Laboratory Procedure Manual: Free T3. University of Washington Medical Center, Dept. of Laboratory Medicine.

What is claimed is:

1. A method for preventing or treating insulin resistance in the cells of a human patient, the method comprising the steps of: a) providing L-tri-iodothyronine in an extended release formulation; and b) administering said extended release formulation to a human patient.

2. The method of claim 1 wherein L-thyroxine is added to the formulation.

3. The method of claim 1 wherein a member of the biguanide class of antidiabetic drugs is added to the formulation.

4. The method of claim 1 wherein a member of the thiazolidinedione class of antidiabetic drugs is added to the formulation.

5. The method of claim 1 wherein a member of the class of endoplasmic reticulum integrated stress response inhibitors is added to the formulation.

6. A pharmaceutical formulation for preventing or treating insulin resistance in a human patient comprising L-tri-iodothyronine, a biguanide, and a thiazolidinedione

7. A pharmaceutical formulation of claim 6 further comprising L-thyroxine.

8. A pharmaceutical formulation of claim 6, wherein the biguanide is omitted from the formulation.

9. A pharmaceutical formulation of claim 6, wherein the thiazolidinedione is omitted from the formulation.

10. A pharmaceutical formulation of claim 6 further comprising a drug which is a member of the class of endoplasmic reticulum integrated stress response inhibitors.

11. A method for preventing or treating insulin resistance in a human patient, the method comprising the steps of: a) providing a drug from the class of endoplasmic reticulum integrated stress response inhibitors in an appropriate formulation; and b) administering said formulation to a human patient.

12. The method of claim 11 further comprising including extended release L-tri-iodothyronine in the formulation.

13. The method of claim 11 further comprising including L-thyroxine in the formulation.

14. The method of claim 11 further comprising including a biguanide in the formulation.

15. The method of claim 11 further comprising including a thiazolidinedione in the formulation.
